(19) United States
(12) Patent Application Publication
(10) Pub. No.: US 2019/0351078 A1
(43) Pub. Date: Nov. 21, 2019

(54) Ultrasound Gel Composition

(71) Applicant: MYT Enterprises LLC, Lakewood, NJ (US)

(72) Inventor: Menachem Treff, Lakewood, NJ (US)

(73) Assignee: MYT Enterprises LLC, Lakewood, NJ (US)

(21) Appl. No.: 16/531,698

(22) Filed: Aug. 5, 2019

Related U.S. Application Data
(63) Continuation-in-part of application No. 15/719,018, filed on Sep. 28, 2017, now abandoned.

(51) Int. Cl. A61K 49/22; A61K 9/00; A61K 9/06; A61K 47/10; A61K 36/886; A61K 47/32

(52) U.S. Cl. CPC A61K 49/226; A61K 9/0014; A61K 47/32; A61K 47/10; A61K 36/886; A61K 9/06

(57) ABSTRACT

A method for conducting an ultrasound session using an ultrasound probe system includes the steps of providing an ultrasound gel for use in combination with the ultrasound probe system, the ultrasound gel defining a composition including 1-10% by weight of Carbopol; 0.001-0.02% by weight of fungistat; 0.001-0.02% by weight of NaOH; 0.001-0.02% by weight of chlorhexidine; 1-5% by weight of glycerin up; 90-95% by weight of purified water; 0.001-0.02% by weight of curacao; 0.1-0.8% by weight of aloe vera leaf juice; 0.01-0.03% by weight of citric acid; 0.001-0.02% by weight of EDTA disodium; 0.001-0.02% by weight of phenoxyethanol; 0.001-0.02% by weight of ethylhexylglycerin; and 0.001-0.02% by weight of methylisothiazolinone; and selectively applying the ultrasound gel on an ultrasound site and operating the ultrasound probe system thereon.

### RELATED APPLICATION

This application is a Continuation application of co-pending U.S. Non-Provisional patent application Ser. No. 15/719,018 filed on Sep. 28, 2017, which claims priority to and incorporates entirely by reference U.S. Provisional Patent Application Ser. No. 62/404,986 filed on Oct. 6, 2016.

### FIELD OF THE INVENTION

This invention relates to ultrasound gels and, more particularly, an ultrasound gel for providing a conductive medium for permitting ultrasound waves to transmit directly to the tissues for imaging, wherein the ultrasound gel is scented and contains a moisturizer for patient comfort and, therefore, ultrasound exam compliance through improved diagnostic accuracy.

### BACKGROUND OF THE INVENTION

Generally, ultrasounds are used in medicine to detect changes in appearance of organs, tissues and vessels, or to detect abnormal masses. Most standard ultrasound gels are primarily composed of propylene glycol and water. Ultrasound gel serves as a conductive medium for enabling a tight bond between the skin and the probe being operated by the medical individual, letting the ultrasound waves transmit directly to the tissues that need to be imaged. As sound waves travel through different materials at varying speeds, the sound waves, after reflecting off structures in the body, bounce back at different intervals depending on the type of material they pass through. The reflected waves are picked up by the probe and relayed to a machine, which displays the distances and intensities of the reflected waves in the form of an image.

Gel is used on all surfaces of the device's head to reduce friction and assist transmission of the ultrasonic waves. The gel is typically squeezed out of a bottle and spread over the patient's skin. The gel is difficult to contain within a desired area of the skin and the thickness of the gel cannot be controlled. Lack of consistent and desired thickness of the gel leads to a less than optimal ultrasound application and, therefore, it is common for an abundance of the gel to be utilized for one procedure.

Use of ultrasound gels is known for being particularly uncomfortable for a variety of reasons, including the cold temperature of gels upon application, as well as the generally pungent odor and sticky residue. In response, many medical facilities use warmers to make their gel a more comfortable temperature before applying it, but the other issues remain. Moreover, there has been much recent focus in the scientific and lay literature on improving the patient encounter and understanding that a more comfortable patient-centric environment not only increases patient satisfaction but improves the quality of the imaging test which in turn can lead to better patient outcomes. When a patient feels comfortable and cared for in the medical setting, they tend to relax and trust the medical professionals who are trying to obtain the best images. This can shorten exam times and ultimately, with better imaging, the radiologist can achieve better diagnostic accuracy. Lastly, patients often complain about the wet residue that remains on the skin until the examination is completed, and the gel is removed.

In light of the problems discussed above, there exists a need for an ultrasound gel provided for increased patient comfort, including an improved viscosity, consistency and skin absorption.

### SUMMARY OF THE INVENTION

In accordance with one form of the present invention there is provided a method for conducting an ultrasound session using an ultrasound probe system, the method including the steps of providing an ultrasound gel for use in combination with the ultrasound probe system, the ultrasound gel defining a composition including 1-10% by weight of Carbopol; 0.001-0.02% by weight of fungistat; 0.001-0.02% by weight of NaOH; 0.001-0.02% by weight of chlorhexidine; 1-5% by weight of glycerin up; 90-95% by weight of purified water; 0.001-0.02% by weight of curacao; 0.1-0.8% by weight of aloe vera leaf juice; 0.01-0.03% by weight of citric acid; 0.001-0.02% by weight of EDTA disodium; 0.001-0.02% by weight of phenoxyethanol; 0.001-0.02% by weight of ethylhexylglycerin; and 0.001-0.02% by weight of methylisothiazolinone; and selectively applying the ultrasound gel on an ultrasound site and operating the ultrasound probe system thereon.

In accordance with another form of this invention invention, there is provided an ultrasound gel composition including carbopol, fungistat, NaOH, chlorhexidine, glycerin up, purified water, curacao, aloe vera leaf juice, citric acid, EDTA disodium, phenoxyethanol, ethylhexylglycerin, methylisothiazolinone, diphenyl oxide, bronyl acetate, fenchyl alcohol, lavandin abrial drome, camphor SYN, isononyl acetate, eucaluptus oil 80%, dihydro myrcenol, terpinyl acetate, DPG, linalyl acetate SYN, and linalools.

In accordance with yet another form of this invention invention, there is provided an ultrasound gel composition including carbopol, fungistat, NaOH, chlorhexidine, glycerin up, purified water, curacao, aloe vera leaf juice, citric acid, EDTA disodium, phenoxyethanol, ethylhexylglycerin, methylisothiazolinone, citronella oil 35%, DPG, geraniol 980, lemon terpene, lemomile, orange oil terpene, pinene alpha and linalyl acetate SYN.

In accordance with yet another form of this invention invention, there is provided an ultrasound gel composition including carbopol, fungistat, NaOH, chlorhexidine, glycerin up, purified water, curacao, aloe vera leaf juice, citric acid, EDTA disodium, phenoxyethanol, ethylhexylglycerin, methylisothiazolinone, menthol, terpineol BP, cineol SP, p-mint arvensis oil stu, carvone (L), menthone (L) and DPG.

### DETAILED DESCRIPTION OF THE PREFERRED EMBODIMENTS

Embodiments of an ultrasound gel composition are disclosed herein and are provided for use in combination with ultrasound devices. Ultrasound gel is typically water-based and contains humectants (water-absorbing and retaining substances) such as glycerin and glycols such as propylene glycol.

Research has indicated that the addition of refreshing scents serves to calm and relax patients, and thereby creates a much more comforting atmosphere for the patient and a more suitable workplace for the medical professional. The selection of scents includes, but is not limited to: Lemon; Lavender; and Mint.

Research has further indicated that children who choose the color of the gel to be used on them allows them to relax them and often takes away the fear of the procedure or use of the ultrasound gel itself. With this in mind, each of the embodiments described herein may include a colorant, such as a florescent colorant.

Applicant has determined that the viscosity and consistency of the ultrasound gel composition disclosed herein is unique in that patients have found it to be particularly smooth while also maintaining a moisturizing effect unlike other ultrasound gel compositions. This unique feature of Applicant's gel composition, wherein the gel remains smooth while also providing a continuous moisturizing effect has proven very beneficial for users of the gel composition in that less gel may be used during an ultrasound as there is less likelihood that the technician would have to reapply additional amounts of gel.

During testing, ultrasound technologists who scan multiple patients throughout the day also found the subtle scent of the ultrasound gel composition not to be overwhelming for the patients. The technologists were the ones who reported the greatest benefit from use of the ultrasound gel composition, as they indicated better scanning abilities due to an improved work environment through the smooth consistency of the ultrasound gel composition. As the aloe component of the gel composition softens the skin and provides a smoother overall consistency, the ultrasound transducer can more effortlessly move along the skin while gently smoothing the skin. As the gel composition remains on the skin, it is absorbed into the skin more quickly than standard gel compositions, and instead of the traditional sticky feeling, the patient's skin feels moisturized.

In initial evaluations of the product, patients were asked about their overall experience with Applicant's gel composition. Those who had a previous ultrasound experience with standard ultrasound gels were most excited by the difference they felt in the gel consistency and, further, they enjoyed the subtle ultrasound scents. The subtle scent was not overwhelming but created an atmosphere of calm and, as previously mentioned, technologists reported enhanced ultrasound scanning abilities in view of an improved work environment as patients were calm due to the pleasant scent and smooth consistency of the product. Patients further noted and appreciated the moisturizing effect of the gel on their skin. Almost all patients indicated that they preferred this gel to standard ultrasound gels.

One embodiment of the present invention is directed to an ultrasound gel formulation including:

5% by weight of Carbopol;

0.01% by weight of fungistat;

0.01% by weight of NaOH;

0.01% by weight of chlorhexidine;

3% by weight of glycerin up;

91.5% by weight of purified water;

0.01% by weight of curacao;

0.4% by weight of aloe vera leaf juice;

0.02% by weight of citric acid;

0.01% by weight of EDTA disodium;

0.01% by weight of phenoxyethanol;

0.01% by weight of ethylhexylglycerin; and

0.01% by weight of methylisothiazolinone.

Another embodiment of the of the present invention is directed to an ultrasound gel formulation including:

5% by weight of Carbopol;

0.01% by weight of fungistat;

0.01% by weight of NaOH;

0.01% by weight of chlorhexidine;

3% by weight of glycerin up;

91% by weight of purified water;

0.01% by weight of curacao;

0.4% by weight of aloe vera leaf juice;

0.01% by weight of citric acid;

0.01% by weight of EDTA disodium;

0.01% by weight of phenoxyethanol;

0.01% by weight of ethylhexylglycerin;

0.01% by weight of methylisothiazolinone

0.01% by weight of diphenyl oxide;

0.01% by weight of bronyl acetate;

0.1% by weight of fenchyl alcohol;

0.4% by weight of lavandin abrial drome;

0.01% by weight of camphor SYN;

0.01% by weight of isononyl acetate;

0.01% by weight of eucaluptus oil 80%;

0.01% by weight of dihydro myrcenol;

0.01% by weight of terpinyl acetate;

0.01% by weight of DPG;

0.01% by weight of linalyl acetate SYN; and

0.01% by weight of linalools.

An additional embodiment of the of the present invention is directed to an ultrasound gel formulation including:

5% by weight of Carbopol;

0.01% by weight of fungistat;

0.01% by weight of NaOH;

0.01% by weight of chlorhexidine;

3% by weight of glycerin up;

91% by weight of purified water;

0.01% by weight of curacao;

0.4% by weight of aloe vera leaf juice;

0.01% by weight of citric acid;

0.01% by weight of EDTA disodium;

0.01% by weight of phenoxyethanol;

0.01% by weight of ethylhexylglycerin;

0.01% by weight of methylisothiazolinone

0.01% by weight of citronella oil 35%;

0.01% by weight of DPG;

0.01% by weight of geraniol 980;

0.25% by weight of lemon terpene;

0.2% by weight of lemomile;

0.01% by weight of orange oil terpene;

0.01% by weight of pinene alpha; and

0.01% by weight of linalyl acetate SYN.

An additional embodiment of the of the present invention is directed to an ultrasound gel formulation including:

5% by weight of Carbopol;

0.01% by weight of fungistat;

0.01% by weight of NaOH;

0.01% by weight of chlorhexidine;

3% by weight of glycerin up;

91% by weight of purified water;

0.01% by weight of curacao;

0.4% by weight of aloe vera leaf juice;

0.01% by weight of citric acid;

0.01% by weight of EDTA disodium;

0.01% by weight of phenoxyethanol;

0.01% by weight of ethylhexylglycerin;

0.01% by weight of methylisothiazolinone

0.45% by weight of menthol;

0.01% by weight of terpineol BP;

0.01% by weight of cineol SP;

0.01% by weight of p-mint arvensis oil stu;

0.01% by weight of carvone (L);

0.01% by weight of menthone (L); and

0.01% by weight of DPG.

In operation, before placing the probe on the skin, the ultrasound gel of the present invention is applied to the skin's surface, so as to provide a conductive medium between the probe and the skin for the traveling and interception of ultrasound waves.

It will be understood that certain features and subcombinations are of utility and may be employed without reference to other features and subcombinations.

While the present invention has been shown and described in accordance with several preferred and practical embodiments, it is recognized that departures from the instant disclosure are contemplated within the spirit and scope of the present invention.

What is claimed is:

1. A method for conducting an ultrasound session using an ultrasound probe system, said method comprising the steps of:
providing an ultrasound gel for use in combination with the ultrasound probe system, the ultrasound gel defining a composition comprising:
(a) 1-10% by weight of Carbopol;
(b) 0.001-0.02% by weight of fungistat;
(c) 0.001-0.02% by weight of NaOH;
(d) 0.001-0.02% by weight of chlorhexidine;
(e) 1-5% by weight of glycerin up;
(f) 90-95% by weight of purified water;
(g) 0.001-0.02% by weight of curacao;
(h) 0.1-0.8% by weight of aloe vera leaf juice;
(i) 0.01-0.03% by weight of citric acid;
(j) 0.001-0.02% by weight of EDTA disodium;
(k) 0.001-0.02% by weight of phenoxyethanol;
(I) 0.001-0.02% by weight of ethylhexylglycerin; and
(m) 0.001-0.02% by weight of methylisothiazolinone; and
selectively applying said ultrasound gel on an ultrasound site and operating the ultrasound probe system thereon.

2. The method as recited in claim 1 wherein said ultrasound gel defines a composition comprising:
(a) 5% by weight of Carbopol;
(b) 0.01% by weight of fungistat;
(c) 0.01% by weight of NaOH;
(d) 0.01% by weight of chlorhexidine;
(e) 3% by weight of glycerin up;
(f) 91.5% by weight of purified water;
(g) 0.01% by weight of curacao;
(h) 0.4% by weight of aloe vera leaf juice;
(i) 0.02% by weight of citric acid;
(j) 0.01% by weight of EDTA disodium;
(k) 0.01% by weight of phenoxyethanol;
(I) 0.01% by weight of ethylhexylglycerin; and
(m) 0.01% by weight of methylisothiazolinone.

3. The method as recited in claim 1 wherein said ultrasound gel defines a composition comprising:
(a) 5% by weight of Carbopol;
(b) 0.01% by weight of fungistat;
(c) 0.01% by weight of NaOH;
(d) 0.01% by weight of chlorhexidine;
(e) 3% by weight of glycerin up;
(f) 91% by weight of purified water;
(g) 0.01% by weight of curacao;
(h) 0.4% by weight of aloe vera leaf juice;
(i) 0.01% by weight of citric acid;
(j) 0.01% by weight of EDTA disodium;
(k) 0.01% by weight of phenoxyethanol;
(I) 0.01% by weight of ethylhexylglycerin;
(m) 0.01% by weight of methylisothiazolinone;
(n) 0.01% by weight of diphenyl oxide;
(o) 0.01% by weight of bronyl acetate;
(p) 0.1% by weight of fenchyl alcohol;
(q) 0.4% by weight of lavandin abrial drome;
(r) 0.01% by weight of camphor SYN;
(s) 0.01% by weight of isononyl acetate;
(t) 0.01% by weight of eucaluptus oil 80%;
(u) 0.01% by weight of dihydro myrcenol;
(v) 0.01% by weight of terpinyl acetate;
(w) 0.01% by weight of DPG;
(x) 0.01% by weight of linalyl acetate SYN; and
(y) 0.01% by weight of linalools.

4. The method as recited in claim 1 wherein said ultrasound gel defines a composition comprising:
(a) 5% by weight of Carbopol;
(b) 0.01% by weight of fungistat;
(c) 0.01% by weight of NaOH;
(d) 0.01% by weight of chlorhexidine;
(e) 3% by weight of glycerin up;
(f) 91% by weight of purified water;
(g) 0.01% by weight of curacao;
(h) 0.4% by weight of aloe vera leaf juice;
(i) 0.01% by weight of citric acid;
(j) 0.01% by weight of EDTA disodium;
(k) 0.01% by weight of phenoxyethanol;
(I) 0.01% by weight of ethylhexylglycerin;
(m) 0.01% by weight of methylisothiazolinone;
(n) 0.01% by weight of citronella oil 35%;
(o) 0.01% by weight of DPG;
(p) 0.01% by weight of geraniol 980;
(q) 0.25% by weight of lemon terpene;
(r) 0.2% by weight of lemomile;
(s) 0.01% by weight of orange oil terpene;
(t) 0.01% by weight of pinene alpha; and
(u) 0.01% by weight of linalyl acetate SYN.

5. The method as recited in claim 1 wherein said ultrasound gel defines a composition comprising:
(a) 5% by weight of Carbopol;
(b) 0.01% by weight of fungistat;
(c) 0.01% by weight of NaOH;
(d) 0.01% by weight of chlorhexidine;
(e) 3% by weight of glycerin up;
(f) 91% by weight of purified water;
(g) 0.01% by weight of curacao;
(h) 0.4% by weight of aloe vera leaf juice;
(i) 0.01% by weight of citric acid;
(j) 0.01% by weight of EDTA disodium;
(k) 0.01% by weight of phenoxyethanol;
(I) 0.01% by weight of ethylhexylglycerin;
(m) 0.01% by weight of methylisothiazolinone;
(n) 0.45% by weight of menthol;
(o) 0.01% by weight of terpineol BP;
(p) 0.01% by weight of cineol SP;
(q) 0.01% by weight of p-mint arvensis oil stu;
(r) 0.01% by weight of carvone (L);
(s) 0.01% by weight of menthone (L); and
(t) 0.01% by weight of DPG.
